(19) United States
(12) Patent Application Publication
(10) Pub. No.: US 2009/0176295 A1
(43) Pub. Date: Jul. 9, 2009

(54) Use of mosses and/or lichens, element and method for reducing the particulate matter content of air

(76) Inventor: Wolfgang Behrens, Ippener (DE)

Correspondence Address: COLLARD & ROE, P.C., 1077 NORTHERN BOULEVARD, ROSLYN, NY, 11576, US

(21) Appl. No.: 11/991,120

(22) PCT Filed: Sep. 8, 2005
(86) PCT No.: PCT/DE2005/001570

§ 371 Date: Feb. 27, 2008

(51) Int. Cl. B01D 53/84; C12M 3/00; A01G 7/00

(52) U.S. Cl. 435/266; 435/289.1; 47/58.1 R

(57) ABSTRACT

The invention relates to the use of mosses and/or lichens as agents for reducing the particulate matter content of air.

The invention relates to a use of mosses and/or lichens, an element and a method for reducing the particulate matter content of air.

In Europe, Council Directive 1999/30/EC of 22 Apr. 1999 set limit values for sulphur dioxide, nitrogen dioxide and oxides of nitrogen, particulate matter and lead in ambient air.

It is already becoming apparent that the measures used until now for observing the limit values set in the Directive for particulate matter are not sufficient.

The definition of particulate matter goes back to the American Environmental Protection Agency's (EPA) “National Air Quality Standard for Particulate Matter” introduced in 1987, also known as the PM Standard. In the American Directive the “PM₁₀ Standard” was defined, for which a limit value has also had to be observed in the European Union since the start of 2005. The designation PM₁₀ means particulate matter with an aerodynamic diameter of less than or equal to 10 micrometres. According to Directive 1999/30/EC, PM₁₀ means particulate matter which passes through a size-selective inlet with a 50% efficiency cut-off at 10 μm aerodynamic diameter.

Particulate matter can originate from both natural and anthropogenic sources. Which source dominates at which location depends on the respective local conditions. The main causes of the anthropogenic proportion of particulate matter in Germany are, for example:
industry: 60 kt/a (35.1%)
private households and small consumers: 33 kt/a (19.3%)
road traffic (without abrasion): diesel engines: 29 kt/a (17.0%)
power stations and district heating plants: 19 kt/a (11.1%)
other traffic: 16 kt/a (9.4%)
transshipment of bulk freight: 8 kt/a (4.7%)
industrial heating: 6 kt/a (3.5%)

In the above-mentioned road traffic proportion, the abrasion of tyres, brake pads and road asphalt is not included.

In urban areas in particular, the proportion of particulate matter emissions from traffic is well over 50 percent.

Cigarette smoke contributes in particular to particulate matter pollution in interior spaces.

Natural sources of dust include:
formation of particulate matter from precursors in the atmosphere
micro-organisms and parts thereof, pollen
rock erosion

In European Directive 1999/30/EC the following limit value legislation is adopted:
1. The daily mean value for PM₁₀ to be observed from 1 Jan. 2005 is 50 μg/m³ with 35 permitted exceedances per calendar year.
2. From 2005 the annual mean value for PM₁₀ is 40 μg/m³.

In order to be able to observe these limit values, different strategies are pursued in the individual European countries: in London a city toll led to 18% less traffic and 12% less particulate matter. In Italy there are vehicle bans. In Austria there are grants for particle filters in diesel vehicles, and biodiesel is promoted. Germany is planning grants for particle filters, an emission-dependent toll for goods vehicles and bans in cities on vehicles not designated low-emission.

Directive 1999/30/EC also specifies that the limit values shall be tightened on 1 Jan. 2010 as follows:
1. The daily mean value for PM₁₀ to be observed is 50 μg/m³ with only 7 permitted exceedances per calendar year.
2. The annual mean value for PM₁₀ is only 20 μg/m³.

Dust is at present essentially blamed for the effects of air pollution on health. These effects range from breathing difficulties, for example coughing, to asthmatic attacks. The extent of the effect of particulate matter on the respiratory tract depends not only on the toxicity of the particles, but also on the size of the particles: the smaller a particle, the deeper into the lungs it can penetrate. PM₁₀ particulate matter reaches the lungs partly because the filtering effect of the nasal and pharyngeal cavities is not sufficient for fine particulate matter with a diameter of less than 10 micrometres. As a result, ultra-fine particles with a diameter of less than 0.1 μm get into the pulmonary alveoli and are removed from there only very slowly or not at all.

The pollution of the air with tiny dust particles is one of today's biggest challenges. Urban areas and areas close to traffic suffer in particular from excessively high particulate pollution. In these areas the annual mean values for particulate matter are above the current limit values all year round, and the daily mean values are also frequently exceeded, sometimes hugely.

The invention is based on the object of providing further measures for reducing the particulate matter content of air.

This object is achieved by a use according to the features of claim 1, with an element according to the features of claim 12 and a method according to the features of claim 33. Developments and advantageous refinements of the invention can be found in the respective subclaims.

The use according to the invention of mosses and/or lichens as means for reducing the particulate matter content of air is based on the understanding that mosses subsist from the atmosphere, in contrast to flowering plants that absorb water and nutrients from earth. Mosses absorb atmospheric water, in particular rain and dew, and the nutrients dissolved therein directly via their surfaces. A further source of nutrients is dust. Dust is held mechanically between the moss leaflets. This especially includes particulate matter. Particulate matter belongs to what is known as floating dust, which owing to its weight and size of <10 μm does not sink and is only precipitated by wet deposition, e.g. during rain or else during the formation of mist droplets. If this particulate matter is brought into contact with mosses by being moved by the wind, it remains suspended between the moss plants.

This filtering effect is on the one hand a purely physical one. This effect could in principle also be achieved by a structure similar to a carpet pile, but on a much smaller scale. In contrast to such artificial surfaces, the moss plants have a hugely enlarged surface area due to their closely arranged leaflets: They are thickly covered with small leaflets. A moss leaflet with an upper and lower side has a surface area of 6 mm². Approx. 80 leaves=480 mm² grow on a 1 cm-long moss plant; approx. 6 moss plants grow on 1 cm, i.e. 2880 mm² per cm² (100 mm²). The surface area enlargement of mosses is therefore almost 30-fold. That is a surface area of 17 280 mm² based on a cubic centimetre.

Mosses that grow on rock, walls and bark in particular accumulate dust over time in this manner, which they hold and whereby their tips grow even more and create more storage space. The particulate matter adhering to a moss leaf can be demonstrated with a scanning electron microscope.

The mentioned filtering effect is on the other hand based on attraction forces owing to different types of charge. It has been found that at least parts of the particulate matter are held on the mosses and lichens or effectively adhere to them as a result of being negatively charged, whereas the mosses and lichens themselves have a positively charged surface. The release by wind or precipitation of the particulate matter held on the surface of the mosses and lichens in this manner is considerably impeded.

The proportion of chemical elements in the particulate matter can be demonstrated in an x-ray spectral analysis.

The invention is furthermore based on the understanding that particulate matter is not only filtered and held, but to a great extent absorbed by the mosses and converted into phytomass. This is true of all inorganic dust such as rock dust. Rock dust is part of the natural particulate matter content of the air but also arises anthropogenically, e.g. by road abrasion. Such mineral particulate matter is chemically bound by ion exchange by the mosses and thereby not only filtered, but also eliminated.

Organic particulate matter is decomposed by bacteria in this biological surface. Investigations using scanning electron microscopy show that bacteria colonise the surface of mosses in large numbers. They decompose the organic compounds that are found there. These organic constituents of the air include not only pollen or spores but also petroleum products or their combustion residues, which are broken down by bacteria. The decomposed products are in turn absorbed by the mosses and converted into phytomass.

Advantageously, mosses also have a chemical filtering effect. The nutrients are absorbed by mosses over their entire surface. In order to be able to absorb these nutrients e.g. from rainwater, the mosses use what is known as ion exchange, to be precise cation exchange. The already mentioned enormous surface areas of mosses mean a high ion exchange capacity and thus a high binding rate of the substances contained in the rainwater.

The capability of mosses to filter heavy metal dust or absorb radionuclides is also based on the principle of ion exchange. Pollutants such as sulphur dioxide dissolved in water in the form of sulphuric or sulphurous acid, and compounds that are damaging to the environment because of their severe fertilising action such as oxides of nitrogen dissolved in water in the form of nitrous or nitric acid, ammonium compounds such as ammonium nitrate or ammonia, are also removed in this manner.

Following the decrease in sulphur dioxide emissions, nitrogen emissions now contribute in particular to air pollution. Nitrogen sources are in particular nitrogen oxides (NOₓ) and ammonia (NH₃). Both compounds are quite unstable and combine in the air, e.g. ammonia combines
with H₂SO₄ to form (NH₄)₂SO₄
with H₂NO₃ to form NH₄NO₃
with OH to form NH₂+H₂O
with HNO₂ to form NH₄NO₂
with HNO₃ to form NH₄NO₃

The resulting compounds (ammonium sulphate, ammonium nitrite, ammonium nitrate) are nitrogen fertilisers such as are used in agriculture. This “air fertiliser” contributes considerably to the eutrophication of our environment, which manifests itself in the disappearance of many plant species in low-nutrient locations and a small number of nitrophilic species taking over. In addition to the reduction in species diversity and the local extinction of plant species, however, whole ecosystems are affected, in particular calcareous neglected grassland, heaths and moors, extensive grassland or rocky summit vegetation.

These nitrogen compounds are however used by mosses as nutrients and absorbed by means of the above-mentioned ion exchange. In this manner the nitrogen compounds are removed from the atmosphere, absorbed and converted into phytomass.

Furthermore, mosses advantageously have an antimicrobial effect.

Pathogenic germs in the form of bacteria or fungal spores are transported through the air not by themselves but bound to relatively large particles such as dust or aerosols. These particles get onto the mosses by dry or wet deposition. These particles are held in the moss by the mechanical filtering effect of the mosses.

For interior spaces this means that bacteria are inhibited from multiplying, that is from dividing, and fungal spores are inhibited from germinating. The interior air is therefore cleaned of germs. In conventional filter substances, a large-scale multiplication of germs can arise as a result, which leads to an epidemic-like spread. Filter systems in air-conditioning systems are particularly affected by this. Owing to the antimicrobial effect of the moss plants, the pathogenic germs cannot multiply in the moss filters. Mosses therefore advantageously lead to a cleaning of the air of disease-causing germs.

A development of the invention provides for the mosses to be selected from the systematic groups of the acrocarpic and pleurocarpic mosses, which consist of the genera Brachythecium, Bryum, Barbula, Funaria, Dicranoweisia, Dicranum, Grimmia, Ceratodon, Homalothecium, Tortula, Abietinella, Hypnum, Rhytidium, Racomitrium and Polytrichum, and preferably to be mixtures of acrocarpic mosses, namely Bryum, Barbula, Dicranoweisia, Dicranum, Funaria, Grimmia, Ceratodon, Polytrichum and/or Tortula, particularly preferably mixtures of pleurocarpic mosses, namely Hypnum, Rhytidium, Brachythecium and/or Homalothecium.

Furthermore it is provided that the mosses are selected from the group consisting of the genera Hypnum, Tortula, Brachythecium and Bryum and are preferably mixtures of Bryum and Tortula, particularly preferably mixtures of Hypnum and Brachythecium.

It has been found that these mosses or mixtures are particularly well suited for achieving the object in interior spaces, that is as interior planting.

According to an advantageous refinement of the invention, the mosses are selected from the group consisting of the genera Ceratodon, Barbula, Bryum and Funaria and are preferably Polytrichum, particularly preferably mixtures of Ceratodon, Barbula, Bryum and Funaria.

It has been found that these mosses or mixtures are particularly well suited for achieving the object outdoors, that is as exterior planting.

In addition, it is provided that the mosses are selected from the group consisting of the genera Abietinella, Hypnum, Rhytidium, Racomitrium, Grimmia and Homalothecium, and are preferably mixtures of Grimmia and Racomitrium, particularly preferably mixtures of Rhytidium, Abietinella and Homalothecium.

It has been found that these mosses or mixtures are particularly well suited for achieving the object in sunny locations outdoors, that is also as exterior planting.

Furthermore it is provided that the mosses are selected from the group consisting of the genera Dicranoweisia, Dicranum, Ceratodon and Bryum and are preferably mixtures of Dicranoweisia and Dicranum, particularly preferably mixtures of Bryum and Ceratodon.

It has been found that these mosses or mixtures are particularly well suited for achieving the object in shady locations outdoors, that is also as exterior planting.

An advantageous refinement of the invention provides for the lichens to be selected from the group consisting of the genera Cladina, Cladonia, Xanthoria, Parmelia, Physcia, Hypogymnia, Peltigera, Evernia, Pseudevernia, Ramalina and Cetraria.

The lichens are advantageously selected from the group consisting of the genera Cladina and Cladonia.

It has been found that these lichens are particularly well suited for achieving the object in interior spaces, that is as interior planting.

The lichens are expediently selected from the group consisting of the genera Cladina, Cladonia, Xanthoria, Hypogymnia and Parmelia, and are preferably mixtures of Xanthoria, Hypogymnia and Parmelia, particularly preferably mixtures of Cladonia and Cladina.

It has been found that these lichens or mixtures are particularly well suited for achieving the object in sunny locations outdoors, that is as exterior planting.

The lichens are advantageously selected from the group consisting of the genera Peltigera, Pseudevernia, Ramalina and Evernia, and are preferably Peltigera, particularly preferably a mixture of Pseudevernia, Ramalina and Evernia.

It has been found that these lichens or mixtures are particularly well suited for achieving the object in humid locations outdoors, that is also as exterior planting.

The lichens are preferably selected from the group consisting of the genera Physcia, Parmelia, Phaeophyscia and Xanthoria, and are preferably mixtures of Phaeophyscia and Parmelia, particularly preferably mixtures of Xanthoria and Physcia.

It has been found that these lichens or mixtures are particularly well suited for achieving the object in areas of high nitrogen pollution outdoors, that is also as exterior planting.

The invention also relates to a element for reducing the particulate matter content of air.

According to the invention, this is a substrate which is applied or can be applied to a horizontal or inclined, artificial or natural surface, on and/or in which mosses and/or lichens or their sprouts and/or spores are arranged and fixed against removal by wind, water or pests.

With an element of this type a reduction of the particulate matter content of the air is achieved, wherein the property of the mosses and lichens of filtering particulate matter out of the air is utilised. Elements of this type can particularly simply be used where the particulate matter content of the air is particularly high and in particular where there is a risk of exceeding the limit values set in Directive 1999/30/EC. This will be primarily in urban areas, in which the pollution by particulate matter is particularly high due to road traffic, or in interior spaces, in which the pollution by particulate matter is particularly high due to cigarette smoke.

The element according to the invention is a biological, living surface. For this reason, such an element has various advantageous properties, which “artificial”, dead, non-living surfaces do not possess. When planted with mosses such elements additionally have specific properties, which elements planted with e.g. sedum, etc. do not have.

The reason for this is the fundamental difference between flowering plants and mosses. Flowering plants are homiohydric plants, that is, they keep their water content constant. They have to keep a positive water balance permanently. They absorb the required water and nutrients from the subsoil via roots. The excess water is released again by transpirational pull through the stomata in the form of water vapour. Succulent leaves allow sedum and similar species to restrict the release of water and adapt it to extreme locations such as roofs so that these plants can survive there. The microclimatically important release of water vapour is therefore minimised. As CAM plants with Crassulaceae Acid Metabolism, these plants close their stomata during the day on dry days in order to avoid excessive losses of water. During the cooler night, carbon dioxide is absorbed into the plant through the now open stomata and stored there in the form of malate. The malate then forms the available source of carbon during the day for assimilation, what is known as the diurnal acid rhythm.

In contrast to flowering plants, mosses have no roots. Instead they absorb water and nutrients through their surface. They can also absorb water vapour at humidities above 80%. With the start of precipitation they become metabolically active and turgescent within seconds. Liquid water is therefore directly and immediately absorbed. Mosses can only assimilate as long as they are moist. They therefore store corresponding amounts of water, which they then slowly release again. The mosses absorb the necessary nutrients from the air. With the absence of roots they absorb the nutrients over their entire surface, either in the form of substances dissolved in rainwater or by means of dust. In this manner they on the one hand filter substances dissolved in rainwater e.g. pollutants out of the air, and on the other hand accumulate dust out of the air both by dry and wet deposition. This takes place by cation exchange. Cations on the surface of the moss leaves are exchanged with hydrogen ions.

As mosses and/or lichens or their sprouts and/or spores have no roots, fixing them against removal by wind, water and/or pests means that the mosses and/or lichens or their sprouts and/or spores remain uniformly distributed on the substrate and are able to develop there.

It is advantageous that mosses and/or lichens or their sprouts and/or spores can manage over a relatively long period without water. They then enter a natural dormant phase. As soon as they come into contact with water and light, growth begins again. This means that the elements do not need intensive care, as the mosses and/or lichens or their sprouts and/or spores survive even during periods of dryness.

In contrast to mosses, flowering plants absorb water through the roots and release the water in the form of water vapour through their stomata. They must therefore be provided continuously with water. In dry locations, for example on roofs, succulents manage by storing water and thereby surviving dry phases.

Mosses are moistened when absorbing water, but dry out during dry phases. In this case they change to a kind of seemingly dead state, what is known as anabiosis. When they are moistened again, they recover their turgescence, sometimes within seconds. Mosses can therefore advantageously survive dry phases of any length in this manner. These dry phases are however not associated with any damage at all. In most species this is associated with a change of growth habit. The mosses lay back and furl their leaves and lose their green colour. Only a few rigid species keep their shape and colour even when dried out. This can be used advantageously as an indicator of when the mosses and/or lichens or their sprouts and/or spores should be artificially watered.

This property also makes it possible, however, to fabricate the elements according to the invention industrially, to transport them easily, that is without moisture, and to store them for a relatively long period.

The growth of the mosses and/or lichens or their sprouts and/or spores only begins when the element according to the invention is applied to a given horizontal or inclined, artificial or natural surface, for example on a roof of a building in an urban area polluted with particulate matter or on a surface in an interior space, and artificially watered.

A development of the invention provides for the substrate to be fixed or to be able to be fixed on the surface by suitable means, preferably by adhesive bonding, by thermal treatment or by mechanical fasteners.

It is furthermore provided for the substrate to include a layer consisting of a water-repellent material, with the layer preferably being a bituminous seal.

The substrate is preferably a vegetation carrier, which includes at least one layer consisting of a water-absorbing and/or storing material consisting preferably of fleece or rock wool.

A considerable increase in the water storage capacity of the element is achieved thereby.

A climate problem in inner-city areas is the rapid seepage loss of precipitated water. Precipitated water is carried away immediately as a result of dense building development, direct discharge of the water into the sewer system and sealing of the ground. The consequence is a distinctly dry urban climate, which is negative in particular because of heat and dust production. The release of water vapour over a relatively long period from the elements according to the invention leads to a clear increase in the relative humidity of the surroundings. The evaporation of large amounts of water that occurs here has a cooling effect on the temperature of the surroundings. In particular, the effects of the urban climate with a dry, hot mesoclimate, particularly in summer, are thereby alleviated. Higher humidities have a positive effect on health and counteract drying of the mucous membranes in the respiratory tract.

The release of water vapour occurs even in dry room air over a period of more than 24 hours.

If the element according to the invention is provided with a water-absorbing and/or storing layer, preferably consisting of fleece or rock wool, the water storage of the mosses can thereby be doubled. This considerably prolongs the period of water vapour release.

According to an advantageous refinement of the invention, the mosses and/or lichens and/or their sprouts and/or spores are arranged directly on the layer consisting of the water-absorbing and/or storing material.

A development of the invention provides for the vegetation carrier to include a hooked layer for fixing the mosses and/or lichens or their sprouts and/or spores against removal by wind, water and/or pests, with the hooked layer preferably lying on the layer consisting of water-absorbing and/or storing material.

The hooked layer is a layer, which is configured in such a manner that it fixes the mosses and/or lichens or their sprouts and/or spores against removal by wind, water and/or pests, thus surrounds the mosses and/or lichens or their sprouts and/or spores in an effectively hook-like manner. The hooked layer is preferably configured as a looped mat. The hooked layer preferably consists of a non-degradable material, particularly preferably of plastic, in particular of nylon or polypropylene. Metal, preferably in the form of a wire mesh, can however also be used as the material.

The hooked layer principle allows the element to be applied also to inclined surfaces such as roofs or walls without the mosses and/or lichens or their sprouts and/or spores sliding off.

In addition, it has the advantage that the mosses and/or lichens are adhered more firmly to the substrate than to a smooth surface.

As the mosses and lichens have no roots, the vegetation is fixed by the hooked layer against removal by wind and water.

Furthermore, birds like to use mosses and lichens as nesting material or search therein for food, which can lead to considerable damage to the vegetation. The hooked layer can considerably restrict the destruction of such moss cover by birds.

Mosses and/or lichens or their sprouts and/or spores are advantageously arranged on and/or in the hooked layer.

To this end, the hooked layer or looped mat is sowed with fragments of moss plants, which develop between the loops, grow into complete plants, and eventually fill or cover the mat completely.

In this method pleurocarpic mosses are preferably used, which have a low-lying, blanket-like growth. In their case, the vertical growth is not so rapid and the risk of erosion lower owing to the horizontal growth. Acrocarpic mosses, which can also be used, on the other hand grow in a cushion- or lawn-like manner. They have a growth rate of a few millimetres per year, but over the course of the year will reach a height which leads to the higher cushions falling out first. The gaps that arise thereby are however closed up again from the side.

The hooked layer is expediently at least partially filled in with a water-storing material.

This enables the water storage of the mosses to be increased.

An advantageous refinement of the invention provides for the vegetation carrier to include a drainage layer, with the drainage layer preferably being arranged beneath the layer consisting of water-absorbing or storing material.

A development provides for the vegetation carrier to include a further layer consisting of a water-absorbing and/or storing material arranged beneath the drainage in order to form condensation.

It is furthermore provided for the vegetation carrier to contain seed of monocotyledonous and/or dicotyledonous plants, which is preferably arranged in the same layer as the mosses and/or lichens or their sprouts and/or spores.

The seed of, for example, succulents, herbs and/or grasses can considerably improve the optical impression of the element.

In addition, according to a development of the invention, the vegetation carrier includes a paper layer, which preferably lies on the mosses and/or lichens or their sprouts and/or spores, and where applicable on the seed of monocotyledonous and dicotyledonous plants, and particularly preferably is itself covered by a hooked layer.

The vegetation carrier expediently contains nutrients, which are preferably introduced into the layer consisting of water-absorbing and/or storing material.

A development provides for the vegetation carrier to include at least one material to improve fire protection.

In this manner the risk of fire can be considerably reduced, in particular during dry phases.

It is further provided for a system for watering the mosses and/or lichens or their sprouts and/or spores, and where applicable the seed of monocotyledonous and dicotyledonous plants to be arranged beneath the body of the vegetation.

The element thereby becomes independent of natural watering and fulfils its task continuously.

It is advantageously provided for the individual layers of the element or its materials to be bound with each other, preferably by lacing.

The element can advantageously be fabricated at the location of use, but preferably at another location, and can be stored and only transported when required at the location of use.

In an advantageous refinement of the invention, the substrate consists of the layer consisting of a water-repellent material and a vegetation carrier according to one of claims 15 to 27 arranged on this layer, preferably firmly bound to this layer, wherein the element can preferably be prefabricated on an industrial scale.

An element is thereby provided, which is easily suited in particular for covering roofs. The prefabrication allows the element to be stored particularly well, to be transported to the location of use and to be particularly simply applied to the given surface. If the layer consisting of a water-repellent material is composed of bituminous sealing sheets, the layer just has to be thermally treated on its underside and placed on the given surface in a conventional manner. The element can be fixed particularly simply in this manner.

It is furthermore provided for the element to be in a non-green state until it is applied to the surface and then watered at the location of use.

The element is preferably configured in the form of a mat and can be rolled up for storage and transport.

According to a development, the surface is a roof, a wall, or a track bed.

The invention additionally relates to a method for reducing the particulate matter content of air, wherein mosses and/or lichens or their sprouts and/or spores are scattered on a given surface and fixed against removal by wind, water and/or being eaten by pests, or an element according to one of claims 12 to 32 is fabricated and fixed to a given surface.

It is furthermore provided for the mosses and/or lichens or their sprouts and/or spores to be watered.

An advantageous refinement of the invention provides for the mosses and/or lichens or their sprouts and/or spores, or the element according to one of claims 12 to 32 to be arranged in an interior space or outdoors on the given surface.

The invention is explained below using an exemplary embodiment, which is shown in the drawing. In the drawing,

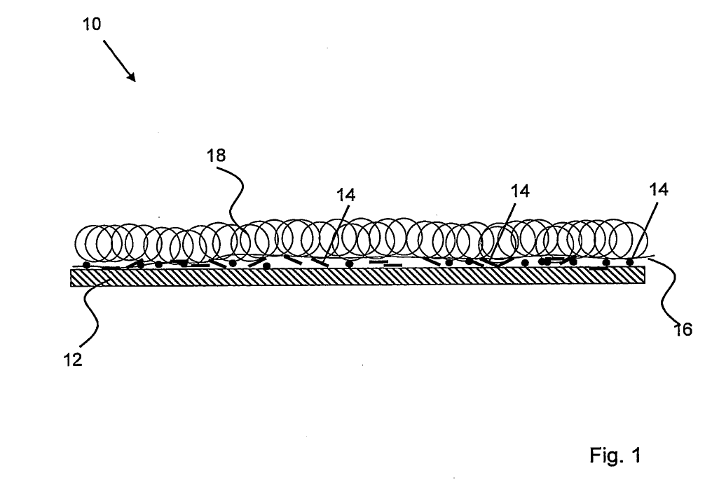

FIG. 1 shows a schematic detail of an element according to the invention in cross section.

The element 10 for reducing the particulate matter content of air shown in FIG. 1 is a mat-like vegetation carrier, which consists of a lower layer consisting of a water-absorbing and storing fleece 12, on which sprouts 14 and/or spores 14 of mosses are uniformly scattered, a paper layer 16, which covers the sprouts 14 and/or spores 14 of the mosses, and a hooked layer 18 in the form of a nylon looped mat arranged on this paper layer 16.

The individual layers 12, 16 and 18 of the element 10 are bound to each other by lacing.

The element 10 can be applied to a horizontal or inclined, artificial or natural surface, in particular to a roof (not shown here). The element 10 is preferably bound to a bituminous sealing sheet, which is arranged on the surface, whereby the bituminous sealing sheet is first thermally treated, thus softened, and the element is then pressed into this soft sealing sheet. Once the sealing sheet has hardened, the element 10 is fixed to the surface. It is possible according to the invention to prefabricate this combination of sealing sheet and element on an industrial scale. Roofs can be covered particularly easily in this manner.

The hooked layer 18 is used for fixing the sprouts 14 and spores 14 of the mosses against removal by wind, water and/or pests.

The element 10 is fabricated on an industrial scale at a different location from the actual location of use and after its manufacture is in an unwatered, non-green state. The element 10 is as a result configured to be very light and can correspondingly be transported easily in a rolled-up form. As mosses and their sprouts and spores enter a dormant phase without water, the element 10 can be stored over a relatively long period of time.

The paper layer 16 prevents the moss sprouts 14 and spores 14 arranged on the fleece layer 12 from falling out through the hooked layer 18 when the element 10 is rolled up or unrolled.

After it has been applied to the said surface at the location of use, the element 10 is artificially or naturally watered, as a result of which the moss sprouts 14 and spores 14 begin to grow. The element 10 then changes to its green state and leads to a reduction in the particulate matter content of the air.

The physiological property of mosses of absorbing water and nutrients from the atmosphere through their surface makes it unnecessary to administer additional nutrients or soil.

### LIST OF REFERENCE SYMBOLS

### Part of Description

10 Element
12 Water-absorbing and/or storing layer
14 Sprouts and spores of mosses
16 Paper layer
18 Hooked layer

1-35. (canceled)

36. A use of mosses and/or lichens as means for reducing the particulate matter content of air.

37. The use according to claim 36, wherein the mosses are selected from the systematic groups of the acrocarpic and pleurocarpic mosses, which consist of the genera Brachythecium, Bryum, Barbula, Funaria, Dicranoweisia, Dicranum, Grimmia, Ceratodon, Homalothecium, Tortula, Abietinella, Hypnum, Rhytidium, Racomitrium and Polytrichum, and are preferably mixtures of acrocarpic mosses, namely Bryum, Barbula, Dicranoweisia, Dicranum, Funaria, Grimmia, Ceratodon, Polytrichum and/or Tortula, particularly preferably mixtures of pleurocarpic mosses, namely of Hypnum, Rhytidium, Brachythecium and/or Homalothecium.

38. The use according to claim 37, wherein the mosses are selected from the group consisting of the genera Hypnum, Tortula, Brachythecium and Bryum and are preferably mixtures of Bryum and Tortula, particularly preferably mixtures of Hypnum and Brachythecium.

39. The use according to claim 37, wherein the mosses are selected from the group consisting of the genera Ceratodon, Barbula, Bryum, Funaria and Polytrichum and are preferably only Polytrichum, particularly preferably mixtures of Ceratodon, Barbula, Bryum and Funaria.

40. The use according to claim 37, wherein the mosses are selected from the group consisting of the genera Abietinella, Hypnum, Rhytidium, Racomitrium, Grimmia and Homalothecium, and are preferably mixtures of Grimmia and Racomitrium, particularly preferably mixtures of Rhytidium, Abietinella and Homalothecium.

41. The use according to claim 37, wherein the mosses are selected from the group consisting of the genera Dicranoweisia, Dicranum, Ceratodon and Bryum and are preferably mixtures of Dicranoweisia and Dicranum, particularly preferably mixtures of Bryum and Ceratodon.

42. The use according to claim 36, wherein the lichens are selected from the group consisting of the genera Cladina, Cladonia, Xanthoria, Parmelia, Physcia, Hypogymnia, Peltigera, Evernia, Pseudevernia, Ramalina and Cetraria.

43. The use according to claim 42, wherein the lichens are selected from the group consisting of the genera Cladina and Cladonia.

44. The use according to claim 42, wherein the lichens are selected from the group consisting of the genera Cladina, Cladonia, Xanthoria, Hypogymnia and Parmelia, and are preferably mixtures of Xanthoria, Hypogymnia and Parmelia, particularly preferably mixtures of Cladonia and Cladina.

45. The use according to claim 42, wherein the lichens are selected from the group consisting of the genera Peltigera, Pseudevernia, Ramalina and Evernia, and are preferably Peltigera, particularly preferably mixtures of Pseudevernia, Ramalina and Evernia.

46. The use according to claim 42, wherein the lichens are selected from the group consisting of the genera Physcia, Parmelia, Phaeophyscia and Xanthoria, and are preferably mixtures of Phaeophyscia and Parmelia, particularly preferably mixtures of Xanthoria and Physcia.

47. An element (10) for reducing the particulate matter content of air, wherein the element (10) is a substrate which is applied or can be applied to a horizontal or inclined, artificial or natural surface, on and/or in which mosses and/or lichens or their sprouts (14) and/or spores (14) are arranged and fixed against removal by wind, water and/or pests, wherein the substrate is a vegetation carrier, which includes at least one layer (12) consisting of a water-absorbing and/or storing material, preferably fleece or rock wool, and a hooked layer (18) for fixing the mosses and/or lichens or their sprouts (14) and/or spores (14) against removal by wind, water and/or pests, wherein the hooked layer (18) preferably lies on the layer (12) consisting of water-absorbing and/or storing material.

48. The element (10) according to claim 47, wherein the substrate is fixed or can be fixed on the surface by suitable means, preferably by adhesive bonding, by thermal treatment or by mechanical fasteners.

49. The element (10) according to claim 47, wherein the substrate includes a layer consisting of a water-repellent material, wherein the layer is preferably a bituminous seal.

50. The element (10) according to claim 49, wherein the mosses and/or lichens or their sprouts (14) and/or spores (14) are directly arranged in the layer (12) consisting of the water-absorbing and/or storing material.

51. The element (10) according to claim 50, wherein the mosses and/or lichens or their sprouts (14) and/or spores (14) are directly arranged on and/or in the hooked layer (18).

52. The element (10) according to claim 50, wherein the hooked layer (18) is at least partially filled in with a water-storing material.

53. The element (10) according to claim 48, wherein the vegetation carrier includes a drainage layer, wherein the drainage layer is preferably arranged beneath the layer (12) consisting of water-absorbing and/or storing material.

54. The element (10) according to claim 53, wherein the vegetation carrier includes a further layer (12) consisting of a water-absorbing and/or storing material arranged beneath the drainage in order to form condensation.

55. The element (10) according to claim 48, wherein the vegetation carrier contains seed of monocotyledonous and/or dicotyledonous plants, which is preferably arranged in the same layer as the mosses and/or lichens or their sprouts (14) and/or spores (14).

56. The element (10) according to claim 48, wherein the vegetation carrier includes a paper layer (16), which preferably lies on the mosses and/or lichens or their sprouts (14) and/or spores (14), and where applicable on the seed of monocotyledonous and/or dicotyledonous plants and particularly preferably is itself covered by the hooked layer (18).

57. The element (10) according to claim 48, wherein the vegetation carrier contains nutrients, which are preferably introduced into the layer (12) consisting of water-absorbing and/or storing material.

58. The element (10) according to claim 48, wherein the vegetation carrier includes at least one material to improve fire protection.

59. The element (10) according to claim 48, wherein the body of vegetation includes on its underside a system for watering the mosses and/or lichens or their sprouts (14) and/or spores (14), and where applicable the seed of monocotyledonous and/or dicotyledonous plants.

60. The element (10) according to claim 48, wherein the individual layers of the element (10) or its materials are bound to each other preferably by lacing.

61. The element (10) according to claim 47, wherein the element (10) can be fabricated at the location of use, but preferably at another location, and can be stored and only transported to the location of use when required.

62. The element (10) according to claim 47, wherein the substrate consists of the layer consisting of a water-repellent material and a vegetation carrier arranged on this layer, preferably firmly bound to this layer, wherein the element can preferably be prefabricated on an industrial scale.

63. The element (10) according to claim 47, wherein the element (10) is in a non-green state until it has been applied to the surface at the location of use and then watered.

64. The element (10) according to claim 47, wherein the element (10) is configured in a mat-like manner and can be rolled up for storage and transport.

65. The element (10) according to claim 47, wherein the surface is a roof, a wall or a track bed.

66. A method for reducing the particulate matter content of air, wherein mosses and/or lichens or their sprouts (14) and/or spores (14) are scattered on a given surface and fixed against removal by wind, water and/or pests.

67. The method according to claim 66, wherein the mosses and/or lichens or their sprouts (14) and/or spores (14) are then watered.

68. The method according to claim 66, wherein the mosses and/or lichens or their sprouts (14) and/or spores (14) are arranged in an interior space or outdoors on the given surface.
